(19) United States
(12) Patent Application Publication
(10) Pub. No.: US 2005/0149073 A1
(43) Pub. Date: Jul. 7, 2005

(54) Mechanisms and methods used in the anastomosis of biological conduits

(76) Inventors: Djavad T. Arani, East Amherst, NY (US); Jack Griffis, Decatur, GA (US)

Correspondence Address: GARDNER GROFF, P.C., 2018 POWERS FERRY ROAD, SUITE 800, ATLANTA, GA, 30339, US

(21) Appl. No.: 10/848,912

(22) Filed: May 19, 2004

Related U.S. Application Data
(60) Provisional application No. 60/530,168, filed on Dec. 17, 2003.

(51) Int. Cl. A61B 17/08

(52) U.S. Cl. 606/153

(57) ABSTRACT

An anastomotic connector for biological conduits comprises a bio-compatible body having an inner surface contour for attaching an outer surface of a biological conduit therein. In another aspect, the present invention is a method of extravascular anastomosis of at least one biological conduit. The method comprises attaching an exterior surface of the biological conduit to an inner surface of an anastomotic connector. In yet another aspect, the invention is a tool for anastomosis and connection of biological conduit(s). The tool comprises a shaft having a sharp tip for penetrating tissue of a biological conduit and an expanding portion for engaging the biological conduit.

### CROSS-REFERENCE TO RELATED APPLICATION

This application claims priority to U.S. Provisional Patent Application Ser. No. 60/530,168, filed Dec. 17, 2003, which is hereby incorporated herein by reference in its entirety.

### FIELD OF THE INVENTION

The present invention relates generally to anastomotic connectors for biological conduits, and more particularly to end-to-side, end-to-end, and side-to-side anastomotic connectors, and to surgical tools for connection of biological conduits.

### BACKGROUND OF THE INVENTION

Biological conduits such as veins and arteries are typically joined in anastomoses in end-to-side, side-to-side, or end-to-end configurations. This is primarily due to the desire to either bypass obstructions of the natural conduit(s), connect damaged conduit(s) and/or replace said conduits. As surgical techniques have improved, and with the introduction of minimally invasive robotic techniques, the minimization of both anastomosis procedure time as well as potential fluid loss have become more important. In addition, the desire to connect or bypass conduits of increasingly small size requires further reduction in surgical difficulty to accommodate the new techniques.

A variety of techniques, devices and technologies have been developed to improve the process described previously, but such techniques and technologies have been limited by many factors, including the size of device, which makes it difficult to be manipulated in the minimally invasive surgical environment; reliance on mechanically complex structures for conduit connection; and introduction of materials to the inside surfaces of the conduits. For these reasons, clinical efficacy has decreased, and the risks associated with man-made materials in direct contact with biological fluid paths have increased.

### SUMMARY OF THE INVENTION

The present invention provides an anastomotic connector for biological conduits. The connector preferably comprises a bio-compatible body having an inner surface contour for attaching an outer surface of a biological conduit therein. In one form, an end-to-side connector preferably comprises a generally tubular portion with a first end comprising an inner circumferential surface for receiving and attaching an outer surface of a biological conduit therein, and a second end comprising an attachment flange extending therefrom.

In a second form, an end-to-side connector preferably comprises first and second conduits with an anastomotic fenestra therebetween. The first conduit has a first end with an inner circumferential surface for receiving and attaching a first biological conduit and a second end in communication with the anastomotic fenestra. The second conduit has first and second ends each with an inner circumferential surface for receiving and attaching a second biological conduit.

In another form, a side-to-side connector preferably has first and second conduits with an anastomotic fenestra providing fluid communication therebetween. Each of the first and second conduits has first and second ends with an inner circumferential surface for receiving and attaching a biological conduit.

In yet another form, an end-to-end connector preferably comprises a generally tubular conduit having first and second ends. Each of the first and second ends comprises an inner circumferential surface for receiving and attaching a biological conduit.

In still another form, a side-to-side connector comprises a first flange with an inner surface contour curved in a first direction and a second flange with an inner surface contour curved in a second direction away from the first direction. An anastomotic fenestra extends through the first and second flanges.

In another aspect, the present invention is a method of extravascular anastomosis of at least one biological conduit. The method comprises attaching an exterior surface of the biological conduit to an inner surface of an anastomotic connector.

In still another aspect, the invention is a tool for anastomosis and connection of biological conduit(s). The tool comprises a shaft having a sharp tip for penetrating tissue of a biological conduit and an expanding portion for engaging the biological conduit.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1A is a perspective view of an end-to-side anastomotic connector according to an example embodiment of the present invention.

FIG. 1B is a side view of the end-to-side connector of FIG. 1A.

FIG. 1C is a sectional view of the end-to-side connector of FIG. 1B taken along line A-A.

FIG. 1D is a detailed view of portion B of FIG. 1C.

FIG. 2 is a perspective view of a tool for anastomosis and connection of biological conduits, according to an example embodiment of the present invention.

FIG. 3A is a side view of the tool of FIG. 2.

FIG. 3B is a side view of the tool of FIG. 2, showing an inflatable member in its expanded state.

FIG. 3C is a top view of the tool of FIG. 3B.

FIG. 3D is a sectional view of the tool of FIG. 3B taken along line C-C.

FIG. 4 is a perspective view of a tool for anastomosis and connection of biological conduits, according to another example embodiment of the present invention.

FIG. 5A is a side view of the tool of FIG. 4, shown with its expansible portion unexpanded.

FIG. 5B is a side view of the tool of FIG. 4, shown with its expansible portion expanded.

FIG. 5C is a top view of the tool of FIG. 4.

FIG. 5D is a sectional view of the tool of FIG. 4, taken along section D-D of FIG. 5C.

FIG. 6A is a top view of the tool of FIG. 4, shown with its head portion retracted against its sleeve portion.

FIG. 6B is a sectional view of the tool of FIG. 4, taken along section E-E of FIG. 6A.

FIG. 6A is an assembly view of an end-to-side anastomotic connector, showing a primary conduit and a graft conduit.

FIG. 7 shows a primary conduit and a graft conduit in end-to-side orientation.

FIG. 8A shows an end-to-side anastomotic connector being placed on a desired connection site for the anastomosis on the primary conduit according to an example embodiment of the present invention.

FIG. 8B shows the end-to-side anastomotic connector of FIG. 8A with its attachment flange secured to the outer surface of the primary conduit according to an example embodiment of the present invention.

FIG. 9A is a side view of the end-to-side anastomotic connector of FIG. 8B.

FIG. 9B is a top view of the end-to-side anastomotic connector of FIG. 9A.

FIG. 9C is a sectional view of the end-to-side anastomotic connector of FIG. 9B taken along line F-F, showing the connector adhered to the outer surface of a primary conduit prior to puncture of the primary conduit.

FIG. 10 shows a puncture tool for anastomosis oriented near an end-to-side anastomotic connector attached to a primary conduit and prior to puncture of the conduit according to an example embodiment of the present invention.

FIG. 11A is an internal cutaway view of the primary conduit having the puncture tool for anastomosis inserted therein.

FIG. 11B is an external perspective view of a primary source conduit punctured by the puncture tool for anastomosis.

FIG. 12A is an internal cutaway view of the primary conduit, showing the expansion of the inflatable member of the puncture tool for anastomosis prior to removal back through the initial puncture site of the primary conduit.

FIG. 12B is an internal cutaway view of the primary conduit, showing the expansion of the inflatable member with placement of the graft conduit within the end-to-side anastomotic connector.

FIG. 13A is an internal perspective view of the puncture tool being removed from the connected assembly with the inflatable member deflated.

FIG. 13B is an external perspective view of the removal of the puncture tool from the connected assembly.

FIG. 14 shows another form of anastomosis tools according to the present invention, used in an end-to-side anastomosis connection.

FIG. 15A is a top cutaway view of the anastomosis connection of FIG. 14, with the tool penetrating through the primary conduit.

FIG. 15B is a sectional view of the anastomosis connection of FIG. 15A, taken along line G-G, prior to expansion of the tool's expansible portion, and with the tool's head portion extended through the primary conduit.

FIG. 15C is a sectional view of the anastomosis connection of FIG. 15A, taken along line G-G, with the tool's expansible portion expanded against the inner wall of the graft conduit, and with the tool's head portion extended through the primary conduit.

FIG. 15D is a sectional view of the anastomosis connection of FIG. 15A, taken along line G-G, with the tool's head portion retracted through the primary conduit to form a circumferential opening in the wall of the primary conduit.

FIG. 16A is a top cutaway view of a completed anastomosis according to an example embodiment of the invention.

FIG. 16B is a sectional view of the completed anastomosis of FIG. 16A, along line H-H, showing the internal circumferential edge of the primary conduit opening attached in abutment with the end face of the graft conduit within the connector.

FIG. 16C is a sectional view, along line H-H of FIG. 16A, of a completed anastomosis according to another example embodiment of the invention, with the internal circumferential edge of the primary conduit opening forming an angled graft attachment with the end of the graft conduit within the connector.

FIG. 16D is a sectional view, along line H-H of FIG. 16A, of a completed anastomosis according to another example embodiment of the invention, with the end of the graft conduit in abutment with the outer surface of the primary conduit adjacent the circumferential edge of the opening.

FIG. 17 is a perspective view of a Y-shaped end-to-side anastomotic connector according to an example embodiment of the present invention, with a lower tubular section for placement along the primary conduit and an oblique upper tube for attachment to an end of the graft conduit.

FIG. 18A is an end view of the Y-shaped end-to-side anastomotic connector of FIG. 17.

FIG. 18B is a sectional view, along line I-I of FIG. 18A, of the Y-shaped end-to-side anastomotic connector.

FIG. 19A is a side view of the Y-shaped end-to-side anastomotic connector of FIG. 17.

FIG. 19B is a sectional view of the Y-shaped end-to-side anastomotic connector, along line J-J of FIG. 19A.

FIG. 20 is a perspective view of an end-to-side anastomotic connector according to another embodiment of the invention.

FIG. 21A is an end view of the end-to-side anastomotic connector of FIG. 20.

FIG. 21B is a sectional view of the end-to-side anastomotic connector of FIG. 21A taken along line K-K.

FIG. 22A is a side view of the end-to-side anastomotic connector of FIG. 20.

FIG. 22B is a sectional view of the end-to-side anastomotic connector, taken along line L-L of FIG. 22A.

FIG. 23 is a perspective view of a side-to-side anastomotic connector according to an example form of the present invention, having a first tubular portion for placement in the primary conduit, and a second tubular portion for placement of the secondary conduit.

FIG. 24A is an end view of the side-to-side anastomotic connector of FIG. 23.

FIG. 24B is a sectional view of the side-to-side anastomotic connector of FIG. 24A taken along line M-M.

FIG. 25A is a side view of the side-to-side anastomotic connector of FIG. 23.

FIG. 25B is a sectional view of the end-to-side anastomotic connector, taken along line N-N of FIG. 25A.

FIG. 26 is a perspective view of an end-to-end anastomotic connector according to an example embodiment of the present invention.

FIG. 27A is an end view of the end-to-end anastomotic connector of FIG. 26.

FIG. 27B is a top view of the end-to-end anastomotic connector of FIG. 26.

FIG. 28A is a side view of the end-to-end anastomotic connector of FIG. 26.

FIG. 28B is a sectional view of the end-to-end anastomotic connector, taken along line O-O of FIG. 28A.

FIG. 29 is a perspective view of a side-to-side anastomotic connector according to another embodiment of the present invention, having a lower flange for attachment to the primary conduit and an upper flange for attachment to the secondary conduit.

FIG. 30A is an end view of the side-to-side anastomotic connector of FIG. 29.

FIG. 30B is a sectional view of the side-to-side anastomotic connector of FIG. 30A taken along line P-P.

FIG. 31A is a side view of the side-to-side anastomotic connector of FIG. 29.

FIG. 31B is a sectional view of the end-to-side anastomotic connector, taken along line Q-Q of FIG. 31A.

### DESCRIPTION OF THE PREFERRED EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

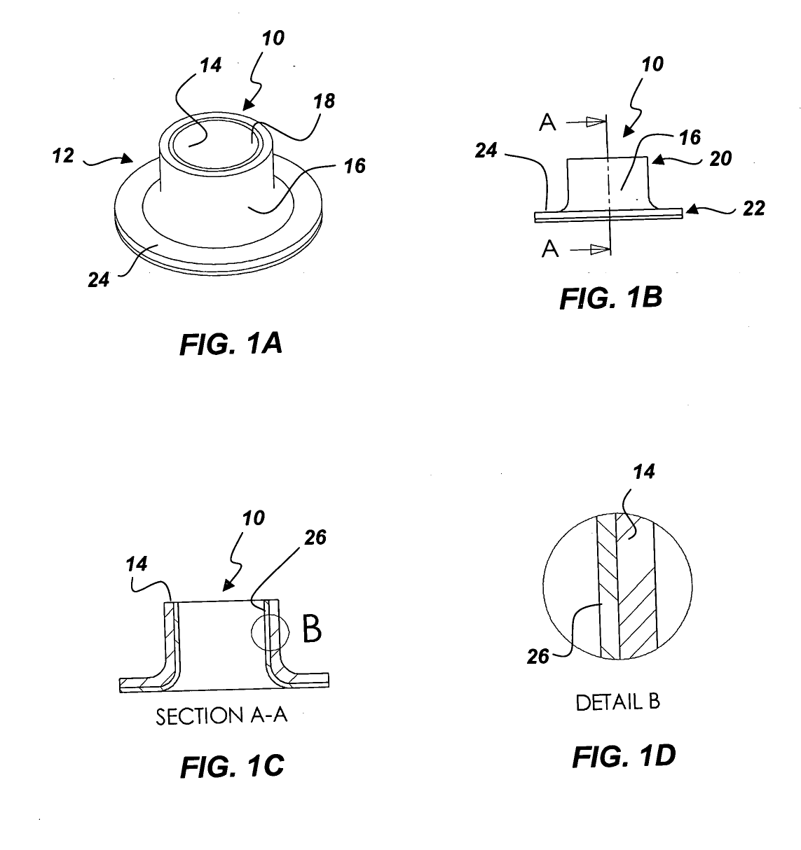

Referring now to the drawings, FIGS. 1A and 1B, as a perspective view and a side view respectively, show an anastomotic connector for biological conduits according to a first example embodiment of the invention, which as a whole is designated by the reference number 10. In this first example embodiment, the anastomotic connector 10 is an end-to-side anastomotic connector 10. The end-to-side anastomotic connector 10 preferably includes a bio-compatible body 12 having a cylindrical inner surface contour 14. In this first example embodiment, the bio-compatible body 12 includes a generally tubular portion 16 and the inner surface contour 14 includes an inner circumferential surface 18 adjacent a first end 20 of the generally tubular portion 16. Extending from a second end 22 of the tubular portion 16 is an attachment flange 24 for attachment to the outer surface of a primary biological conduit such as a vein, an artery, a lumen, a duct, or other organ or vessel. The inner circumferential surface 18 of the tubular portion 16 is preferably configured to receive and attach to the external surface of an end of a secondary or “graft” biological conduit. A bio-compatible adhesive 26 can be applied along at least a portion of the inner surface 18, and/or the connection face of the flange 24, as depicted in FIGS. 1C and 1D. It should be noted that in addition to the bio-compatible adhesive 26, the anastomotic connector 10 can be sutured, stapled or otherwise attached to the external biological conduits.

Preferably, the anastomotic connector 10 is made of a bio-compatible polymer that is flexible in nature, provides a more natural connection between biological conduits, and accommodates movement of the internal biological structures to prevent potential obstruction of the anastomosis due to conduit shifting. For example, the anastomotic connector 10 can include a variety of solid or fabric-like materials such as polytetrafluoroethylene (PTFE), expanded polytetrafluoroethylene (ePTFE), elastomers such as urethanes and silicone, or polyetherteraphalate (PET) compatible with the surrounding tissues of the conduits for anastomosis. The materials of construction are preferably translucent or transparent, which advantageously allows for the use of photopolymerizing adhesives. Translucent or transparent materials of construction also are preferable because the surgeon can more easily see the placement of the biological conduits within the anastomotic connector 10. The attachment surfaces of the solid or fabric-like structures are preferably impregnated or coated with the bio-compatible adhesive 26, which can be activated by a variety of means, including through contact with a biological fluid flowing through the area, photopolymerization by ultra-violet or infra-red light exposure, or activation through microcapsules of adhesive and adhesive activators that rupture due to expansion of, or surface pressure on, the connector 10 during anastomosis.

The method of manufacture of anastomotic connectors 10 according to the present invention can take a variety of forms including injection molding, blow molding, vacuum molding, dipping, extrusion, or weaving. Injection molding of the components allows for low-cost, high volume manufacture in a non-complex format, such as end-to-side connectors 10 of perpendicular conduits. Blow molding can be utilized to form complex tubular structures using extruded tubes within a mold shaped for the outside form of the connector 10. This process would also apply for vacuum molding. Dipping of materials over a preformed shape would also be applicable for manufacture, especially for assembly of the outside structure over a pre-form with the bio-compatible adhesive 26 applied to the outer surface.

Extrusion would be applicable for the forming of the various parallel tubular portions 16, which would then be adhered or thermally bonded to one another during secondary processing. However, extrusion of dual or multi-lumen tubular portions 16 that are connected is also a potential manufacturing technique, with removal of plastic at the relative location of the anastomotic fenestra being a post-processing step. In addition, compatible materials of varying durometers can be utilized in assembly, allowing for closer matching to the flexibility of the desired biological conduit for anastomosis.

All materials listed above are readily adaptable to each of these processes, with Dacron (polyetherteraphalate in thread form) primarily being utilized for weaving over a pre-form of the inside geometry. Application of the bio-compatible adhesive 26 to these assemblies can be achieved in a variety of ways, including painting the inner surface contour 14 of the connector 10, incorporating the adhesive 26 into the dipping solution for forming of the internal layers of the connector 10, pre-coating the internal form used for molding such that the external structure adheres to a pre-placed internal adhesive layer, or storage of the adhesive 26 separate to the connector 10 and application of the adhesive 26 to the inner surface contour 14 intended for fixation to the conduits during the surgical anastomosis.

Exemplary adhesives 26 include gelatin-formaldehyde-resorcinol type glue, photoethyleneglycol diacrylate, thrombin and fibrin microcapsules, and methyl and butyl cyanoacrylates. Photopolymerizing glues are of especial interest due to the ability to shift or manipulate the conduits prior to final fixation using light.

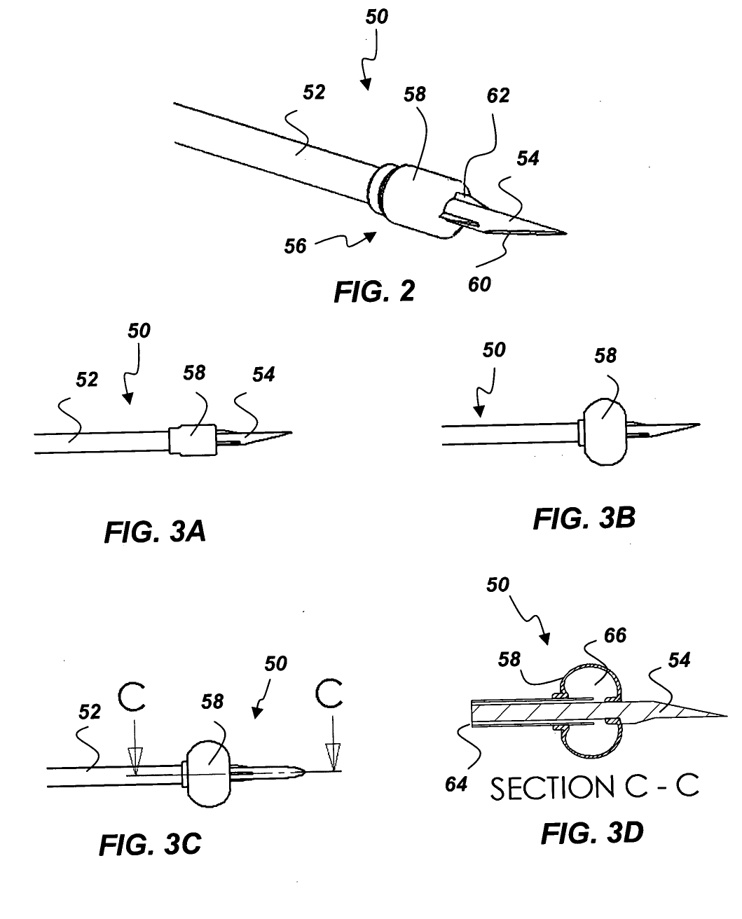

FIG. 2 shows an example embodiment of a tool 50 for anastomosis and connection of biological conduit(s). The tool 50 preferably includes a shaft 52 having a sharp tip 54 for penetrating the tissue of a biological conduit, and an expanding portion 56 for engaging the inner surface of the biological conduit. The shaft 52 is optionally flexible and/or steerable. The tip 54 preferably includes a trocar style tip 60 and optionally one or more radial or helical side cutting blades 62, as depicted in FIG. 2. The blades 62 can be longitudinally oriented along the outer surface of the tip 52 such that the penetration of the tip 54 into the biological conduit produces a multitude of radial cuts.

As depicted in FIG. 2, the expanding portion 56 is positioned proximate the tip 54. The expanding portion 56 preferably comprises an inflatable member 58, such as a balloon, that can be inflated or otherwise expanded. FIG. 3A shows a side view of the tool 50, with the inflatable member 58 in its deflated state, and FIG. 3B shows a side of the tool 50 having the inflatable member 58 in its inflated state. Expansion of the inflatable member 58 of the tool 50 can be accomplished, for example, through balloon expansion in the manner of a typical balloon catheter, such as by use of a syringe, a pump, or though other means of fluid or mechanical expansion, including pneumatically using a gas or hydraulically using a liquid. As shown in FIG. 3D a lumen 64 extends through the shaft and communicates expansion fluid from an external pressure source to the inflatable member 58.

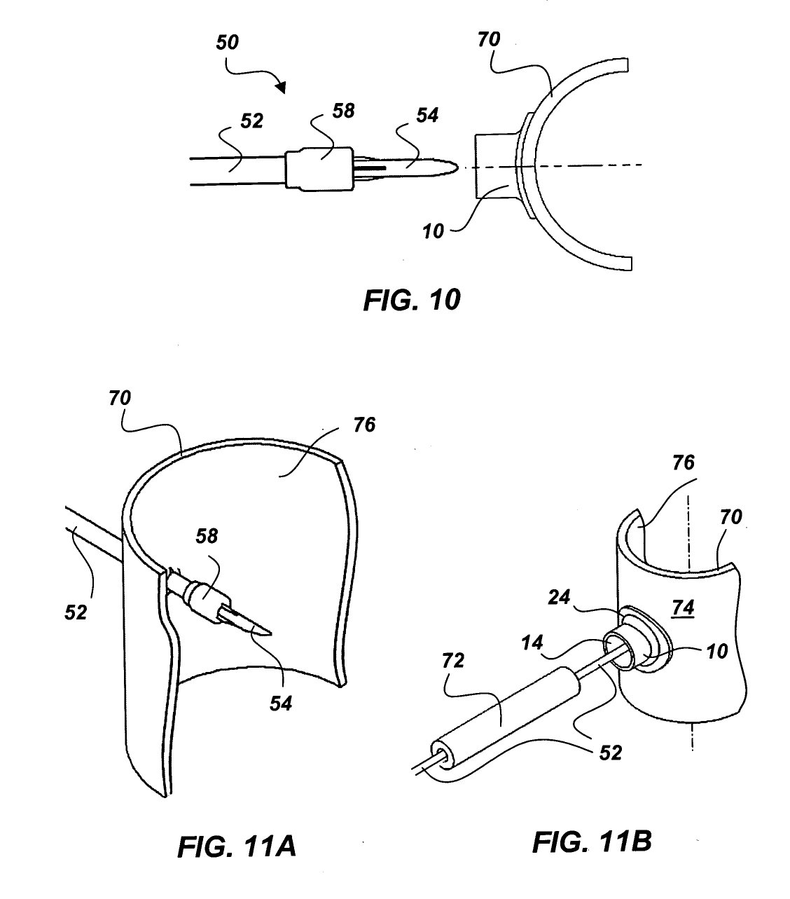

Expansion of the inflatable member 58 serves to secure the position of the tool 50 within the biological conduit, and/or to press the biological conduit against the anastomotic connector to form adhesive contact and complete the connection therebetween, as described below with reference to FIGS. 10-13. In alternate forms of the invention, positioning and adhesive contact are implemented through other methods, including mechanical compression, mechanical expansion of the puncture hole, or interference with the puncture hole due to increase of diameter of the tool 50 as it passes through the target conduit wall (i.e., a tapered section of the tool).

Materials used to construct the tool 50 for creation of the anastomotic fenestra can utilize a variety of bio-compatible polymers or metals and metal alloys. The shaft 52 of the tool 50 can include a variety of solid materials such as metals, metal alloys and polymers. Metals and metal alloys such as titanium (Ti), stainless steel (SS) and nickel titanium alloy (NiTi) are suitable for use in example embodiments. Polymers such as polycarbonate (PC), polyimide, polyetherimide, liquid crystal polymer (LCP), polyetheretherketone (PEEK), polyamide (Nylon), polyamide blends, high density or ultra high molecular weight polythethylene (HDPE, UHMWPE) and polyetherteraphalate (PET) are also suitable for use. The tip 54 of the tool 50 can utilize a variety of materials as well, but would preferably be formed from metals and metal alloys such that the tip 54 can be sharpened or ground to provide easy insertion into the target conduit for bypass through its outer wall. Stainless steel is especially well adapted to this indication. The inflatable member 58 of the tool 50 can be formed by a variety of materials allowing it to have a reduced deflated profile during insertion into the target conduit and limiting the potential damage to the wall during placement. Materials compatible with this feature include polyethylene (PE), polyamide (Nylons), polyether block amides (PEBAX) elastomers such as silicone, urethanes and latex, or polyetherteraphalate (PET) and polyetherteraphalate blends (such as Hytrel). Inflation of the inflatable member 58 can utilize either liquid or gas fluid.

In some embodiments, the tool 50 may further include fiber optics for a light source for photo-polymerization or imaging. If a photopolymerizing adhesive 26 is used, then an ultra-violet or infra-red light source can be positioned near the tip 54 of the tool 50 so as to activate the adhesive 26. The tool 50 may also incorporate one or more irrigation and/or flushing lumens, and/or lumen(s) for delivery of accelerants or catalysts for adhesive activation. Alternatively or additionally, the tool 50 may include an endoscopic imaging means such as fiberoptics or a CCD camera, and/or internal viewing ports near the tip 54 to aid in the guidance and placement of the tool 50.

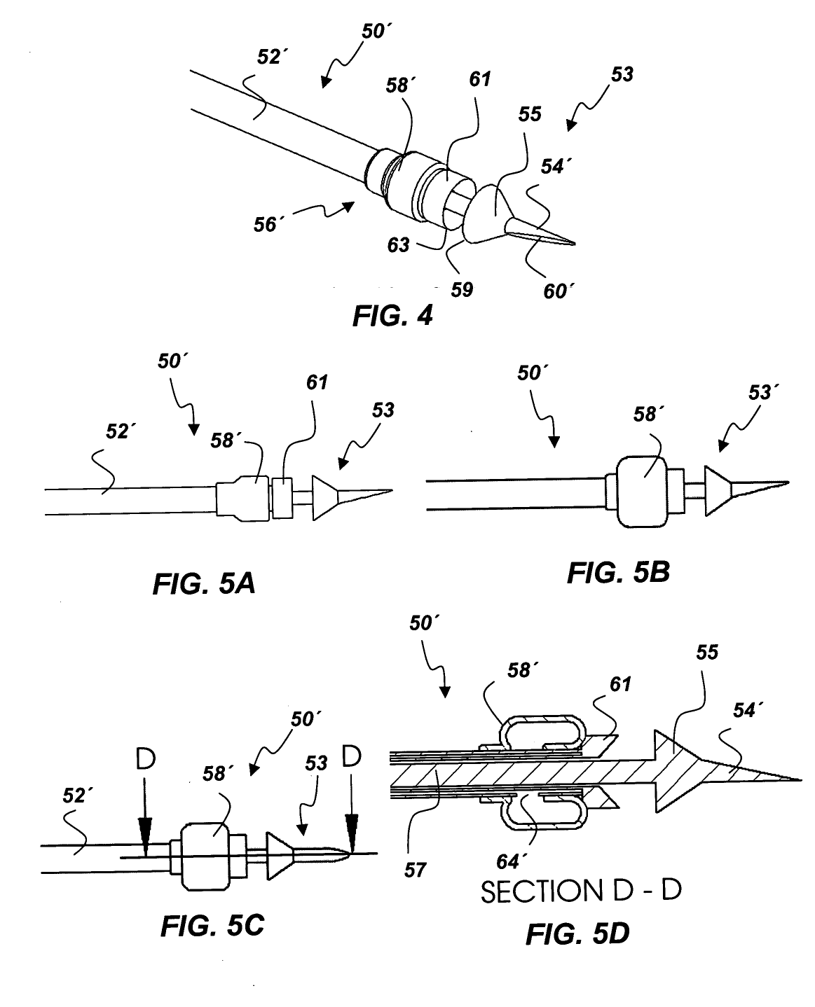

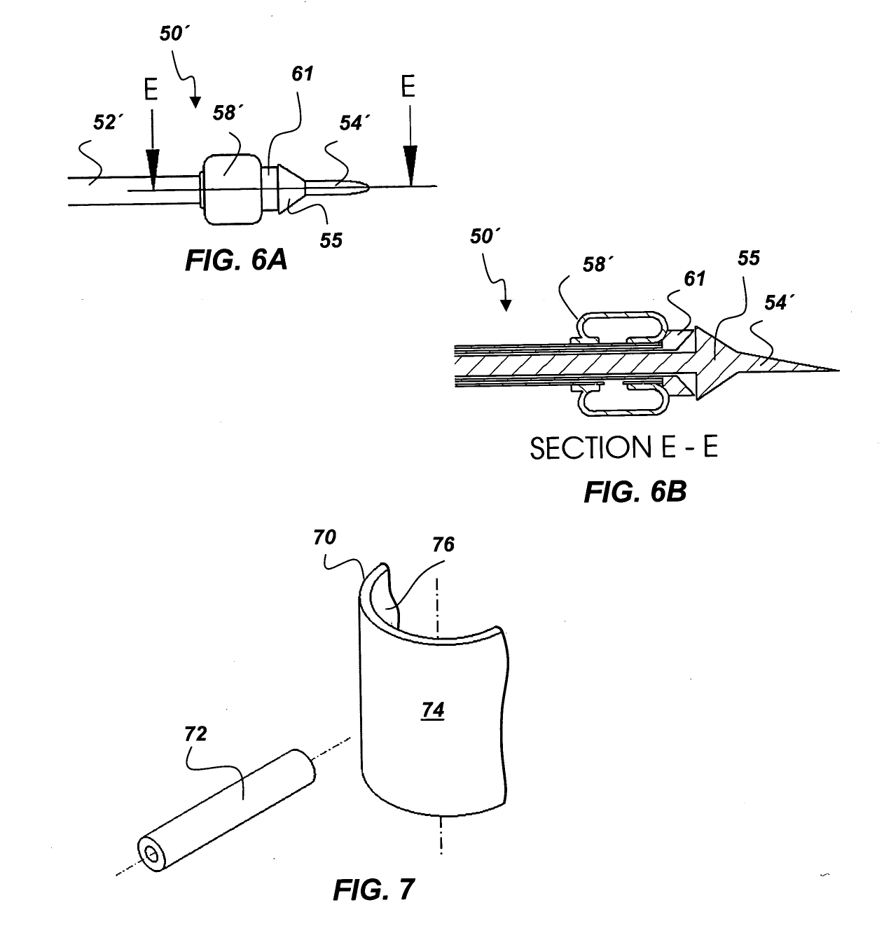

FIGS. 4-6 illustrate another example embodiment of a tool 50′ for anastomosis and connection of biological conduit(s). Similar to the first embodiment, the tool 50′ includes a shaft 52′ and an expanding portion 56′ having an inflatable member 58′ for engaging the biological conduit. The shaft 52′ preferably comprises an extendable and retractable cutting head 53, having a sharp tip 54′ with a trocar blade 60′ for penetrating tissue of a biological conduit, a radial cutting portion 55 for removing tissue. An inner shaft 57 is attached to the cutting head 53, and slides within a channel or lumen extending through the shaft 52′ to extend and retract the cutting head relative to the expanding portion 56′ of the shaft 52′. Optionally, the inner shaft 57 and/or the outer shaft 52′ are flexible and/or steerable for improved guidance. The cutting head 53 preferably comprises a radially flared portion 55, having a generally conical taper extending rearward from the tip 54′. The tool 50′ preferably also includes a cutting collar 61 mounted to the outer shaft 52′, between the expanding portion 56′ and the cutting head 53. The collar 61 preferably has an edge 63 that cooperates with a confronting edge 59 of the cutting head 53 to incise tissue. For example, as the cutting head is advanced to penetrate the tissue of a biological conduit, the tip 54′ and flared portion 55 extend through the wall of the conduit, and the tissue of the conduit wall is captured between the edges 63 and 59 of the collar 61 and cutting head 53 respectively. When the head 53 is retracted by drawing the inner shaft 57 through the outer shaft 52′, the edges 59 and 63 are pulled together, as depicted more clearly in FIGS. 6A and 6B, to incise a circular (or otherwise shaped) circumferential opening through the tissue of the conduit wall. Both of the edges 59, 63 may be sharpened, or one of the edges can surface against which the other cuts. Preferably, as the tool is removed, the collar 61 positively retains the excised biological tissue for removal from the body.

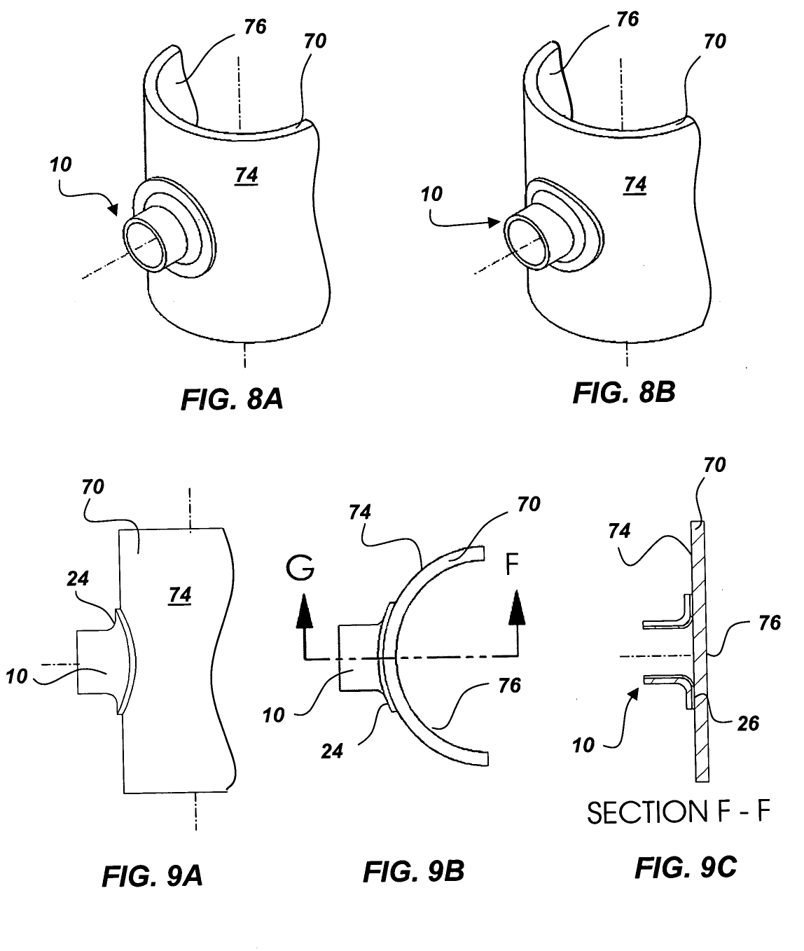

FIG. 7 illustrates the relative, perpendicular orientation between a primary conduit 70 and a secondary or graft conduit 72. The primary conduit 70 has an outer surface 74 and an inner surface 76. An end-to-side anastomotic connector 10 can be positioned at a desired location on the outer surface 74 of the primary conduit 70 as shown in FIG. 8A. The bio-compatible adhesive 26 can be applied to the attachment flange 24 of the anastomotic connector 10 and affixed to the outer surface 74 of the primary conduit 70, so as preferably to provide a leak-proof seal, as depicted in FIG. 8B. The connector 10 can additionally or alternatively be sutured, stapled or otherwise attached to the conduit 70 as desired.

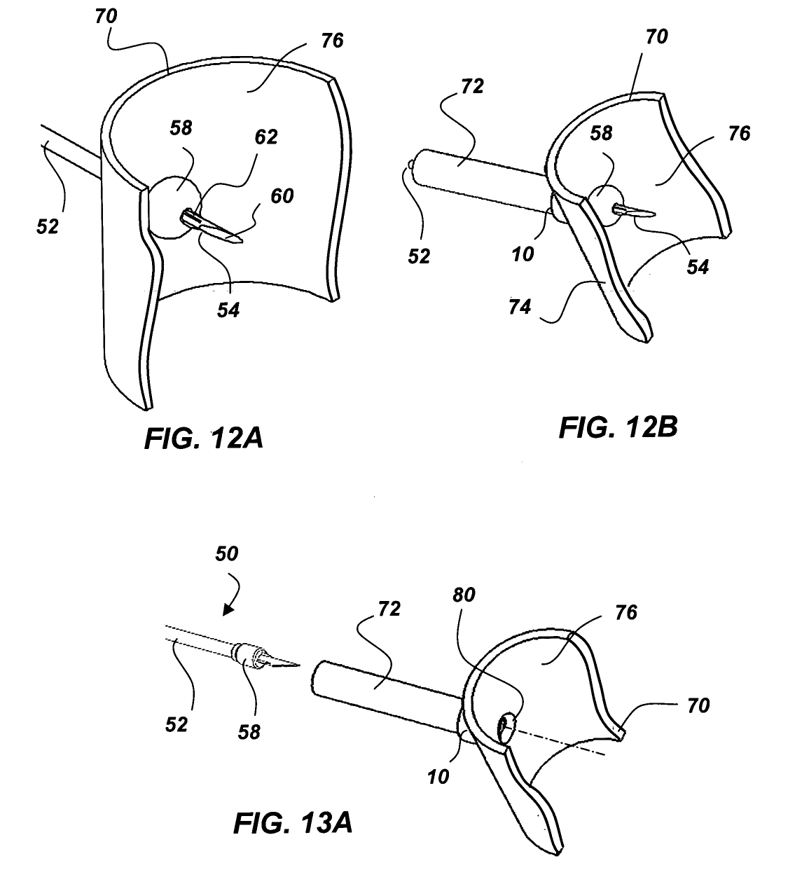

FIGS. 9A-9C show side view and sectional views of the anastomotic connector 10 affixed to the outer surface 74 of the primary conduit 70 prior to creation of an anastomotic fenestra, or fluid pass-through. To create an anastomotic fenestra, the tool 50 is oriented perpendicularly to the primary conduit 70 with the tip 54 positioned to penetrate the outer surface 74 of the primary conduit 70, as depicted in FIG. 10. The tool 50 is then placed through the graft conduit 72 and utilized to puncture the primary conduit 70 through the inner surface 76 as shown in FIGS. 11A and 11B. The expanding portion 56 of the tool 50 is passed into the internal space of the primary conduit 70 and inflated to press the outer surface of the conduit against the adhesive of the connector's flange, resulting in a leak proof compressive seal between the primary conduit and the connector, as shown in FIG. 12A. The expanding portion 56 is expanded within the graft conduit in similar fashion to contact the outer surface of the end of the secondary conduit against the adhesive of the interior of the connector's tubular body portion and complete the connection. FIG. 12B shows the placement of the graft conduit 72 within the anastomotic connector 10 and fixation prior to removal of the tool 50.

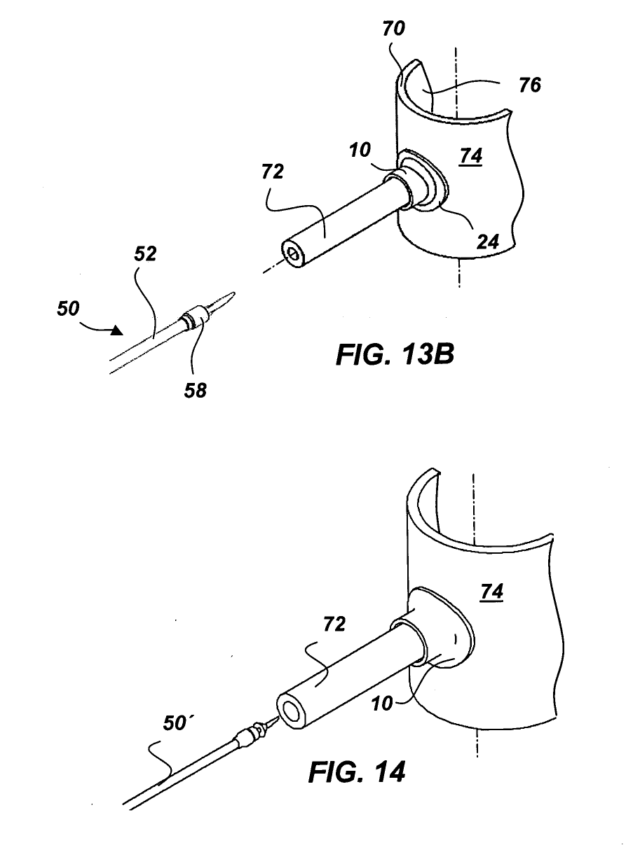

FIG. 13A shows an interior view of the primary conduit 70 with the expanding portion 56 of the tool 50 deflated and removed from the graft conduit 72. FIG. 13B is an external view showing the removal of the tool 50 after attachment of the graft conduit. FIG. 14 is a view of the removal of tool 50′. Removal of the tool 50 can also occur with the inflatable member 58 in a fully or partially expanded state, to promote contact and adhesion of the graft conduit 72 to the inner surface contour 14 of the anastomotic connector 10 as well as adhesion of the connector flange to the outer surface 74 of the primary conduit 70 around the anastomotic fenestra 80.

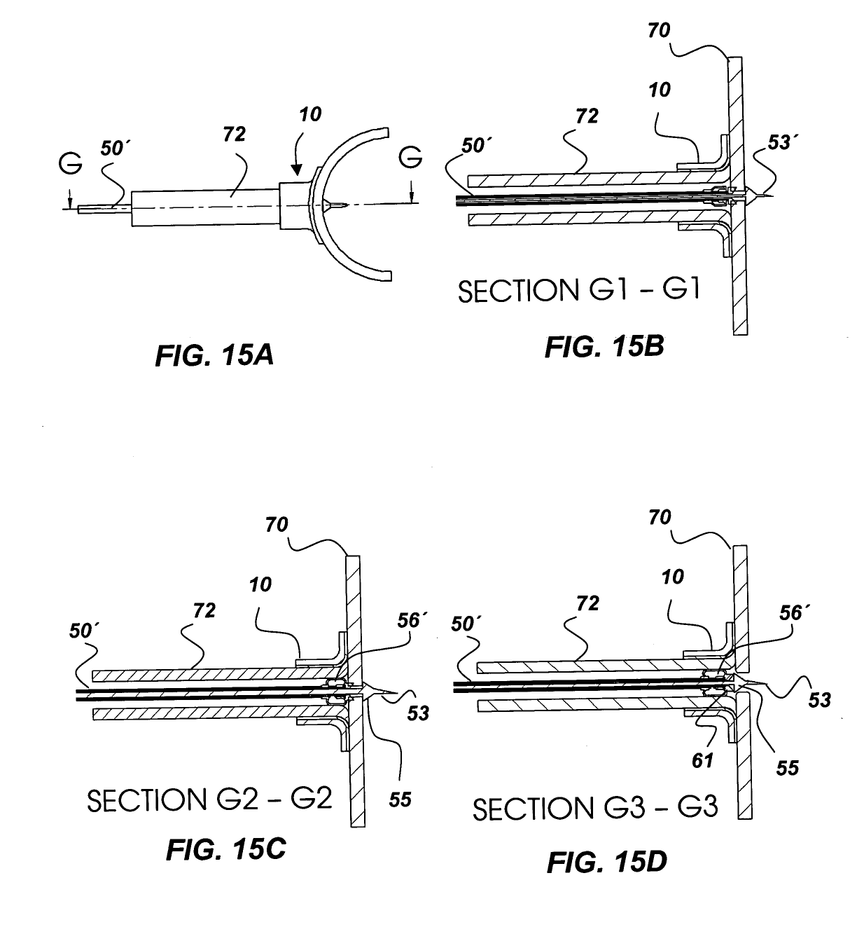

FIG. 15A displays a top view of a nearly completed anastomosis and showing an alternative embodiment of the puncture tool 50′ located within the central portion of the primary conduit. As depicted in more detail in FIG. 15B, a sectional view of the anastomosis of FIG. 15A is shown taken along line G-G. The puncture tool 50′ is shown extending through the wall of the primary conduit, with the conduit wall captured between the flared cutting head and the cutting collar. FIG. 15C is a sectional view of the anastomosis of FIG. 15A taken along line G-G showing the inflatable member 58′ in its inflated state, to secure the outer shaft of the tool in place within the secondary conduit. FIG. 15D is a sectional view of the anastomosis of FIG. 15A taken along line G-G when the radial cutting portion 55 of the tool 50′ is retracted from the primary conduit by pulling the inner shaft back relative to the outer shaft, which draws the confronting edges of the cutting head and the cutting collar into engagement, and results in a circumferential cut through the wall of the primary conduit.

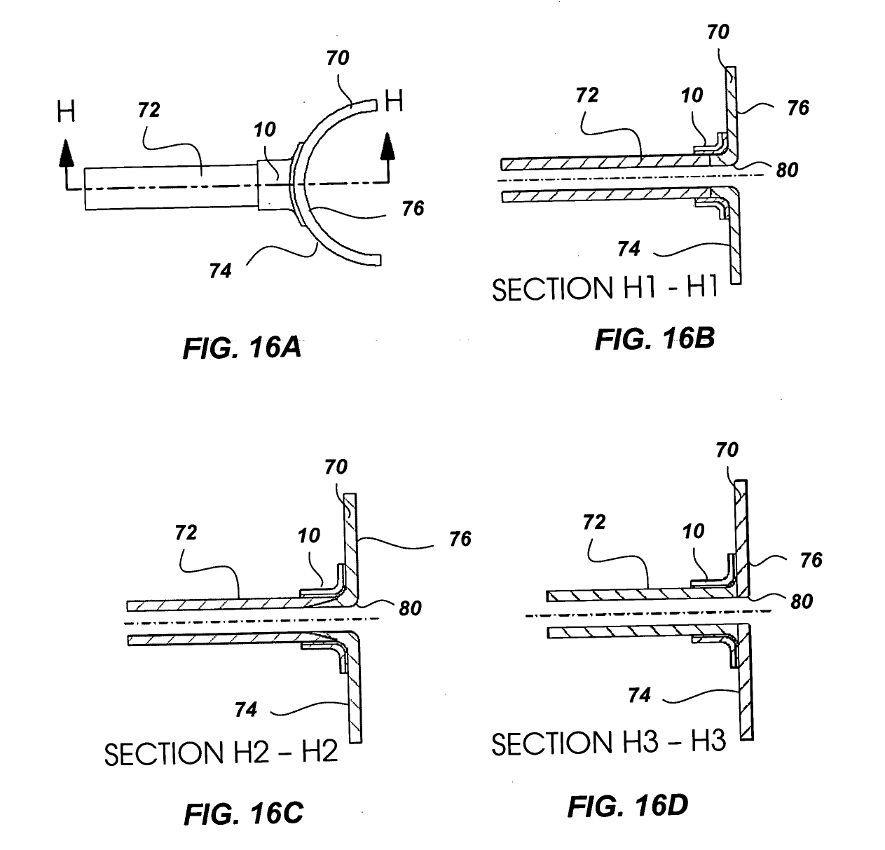

FIG. 16A shows a top view of a completed anastomosis using the end-to-side connector 10. FIG. 16B displays a sectional view of one embodiment of the completed anastomosis of FIG. 16A, wherein the tool 50 was removed while its inflatable member 58 was in at least a partially expanded state. The end of the graft conduit 72 was cut at a 90° angle and attached to the connector 10. Alternatively, the graft conduit 72 can be adhered to the inner surface contour 14 of the anastomotic connector 10 such that it is flush with the outer surface 74 of the primary conduit 10 as shown in FIGS. 16C and 16D. The graft conduit 72 can be clamped to prevent leakage through the anastomotic connector 10 prior to a secondary anastomosis of the graft conduit 72 as a bypass (not illustrated).

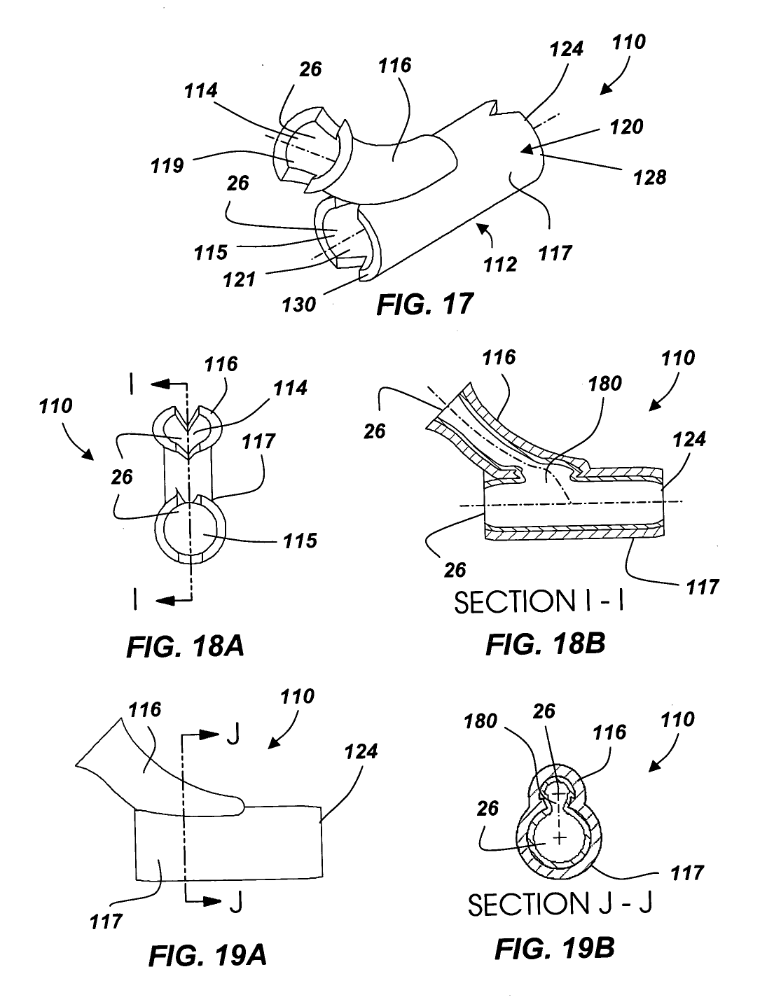

FIGS. 17-31 depict alternate embodiments of the anastomotic connector 10 of FIG. 1. For example, FIG. 17 depicts an alternative embodiment of an end-to-side anastomotic connector 110. This anastomotic connector 110 provides for a non-perpendicular anastomosis (Y-shaped as shown) of the graft conduit to the primary conduit. The anastomotic connector 110 includes a bio-compatible body 112 having a first tubular portion 116 and a second tubular portion 117. The tubular portions 116, 117 have inner surface contours 114, 115, respectively, with the bio-compatible adhesive 26 applied thereon for connecting to the external surfaces of ends of the graft conduit and the primary conduit.

Preferably, the first tubular portion 116, illustrated in FIGS. 18A & 18B, comprises a closed end 124 in communication with the interior of the second tubular portion, and an open end 126 for the graft conduit. The inner surface contour 114 includes an inner circumferential surface 118 adjacent the open end 126 of the generally tubular portion 116, for receiving and attaching a graft conduit. The distal end of the graft conduit would preferably be cut at an angle to assist in the non-perpendicular connection to the primary conduit.

The second tubular portion 117 preferably has a first open end 128 and a second open end 130. The inner surface contour 115 includes inner circumferential surfaces 119, 120, and 121 adjacent the open ends 126, 128, and 130 of the second tubular portion 117. The inner circumferential surfaces 119, 120 are configured to receive and attach ends of a primary conduit. The primary conduit may be placed substantially parallel to the graft conduit through the second tubular portion 117. An anastomotic fenestra 180 is preferably provided for fluid communication between the interior channels of the tubular portions 116, 117 as shown in FIG. 18B. Preferably, the open ends 126, 128, and 130 of the tubular portions 116, 117 are flared to ease the placement of the graft and primary conduits within the anastomotic connector 110 prior to fixation of the conduits with the adhesive 26. This embodiment is preferable for providing easier connections of the graft conduit to the primary conduit when the graft conduit is cut at an angle other than 90° for anastomosis through the closed end 124.

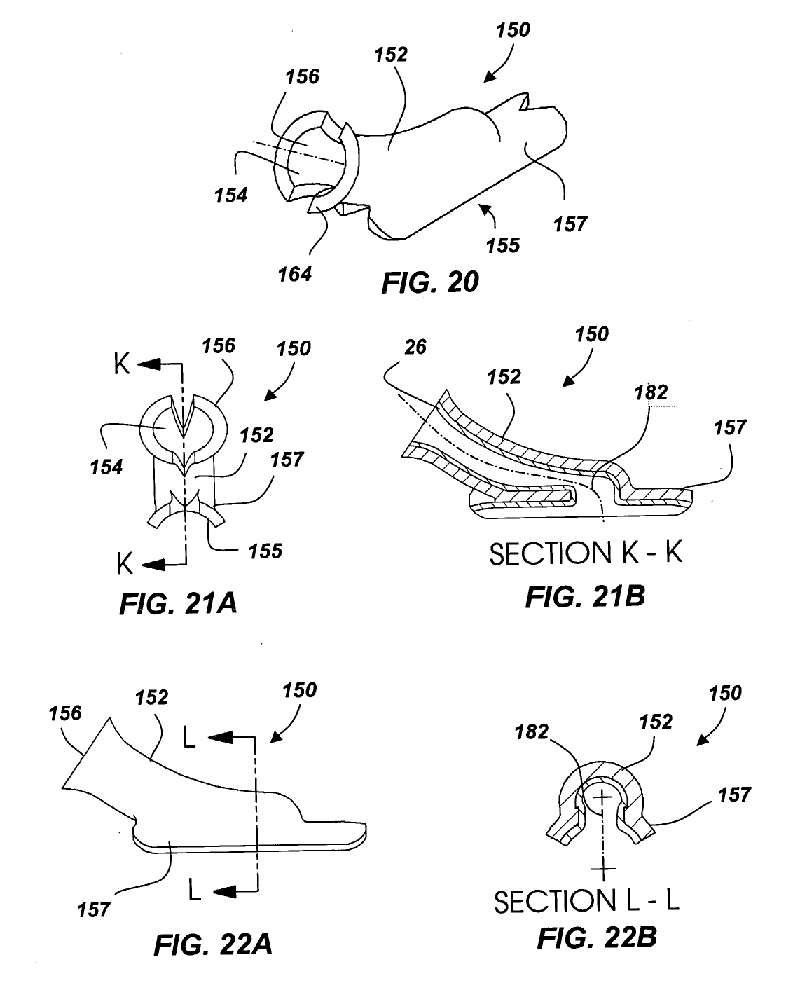

Another example embodiment of an end-to-side anastomotic connector is depicted in FIG. 20. This anastomotic connector 150 provides for a non-perpendicular anastomosis (Y-shaped as shown) of the graft conduit to the primary conduit. The end-to side anastomotic connector 150 includes a bio-compatible body 152 having a first tubular portion 156 and an open flange 157. The tubular portion 156 and the flange 157 have inner surface contours 154, 155 with the bio-compatible adhesive 26 applied thereon for connecting to the graft conduit and the primary conduit.

Preferably, the first tubular portion 156, illustrated in FIGS. 21B & 22A, provides an open end 164 for receiving and attaching an end of the graft conduit. Preferably, the open end 164 of the tubular portion 156 is flared to ease the placement of the conduit within the anastomotic connector 150 prior to fixation of the conduits with the adhesive 26. The inner surface contour 154 includes an inner circumferential surface 158 adjacent the open end 164 of the generally tubular portion 156 for receiving and attaching a graft conduit. The distal end of the graft conduit would preferably be cut at an angle to assist in the non-perpendicular connection to the primary conduit. The primary conduit may be placed substantially parallel to the graft conduit and attached to the flange 157. An anastomotic fenestra 182 is preferably provided between the tubular portion 156 and the flange 157 as shown in FIG. 21B for fluid communication between the primary and graft conduits.

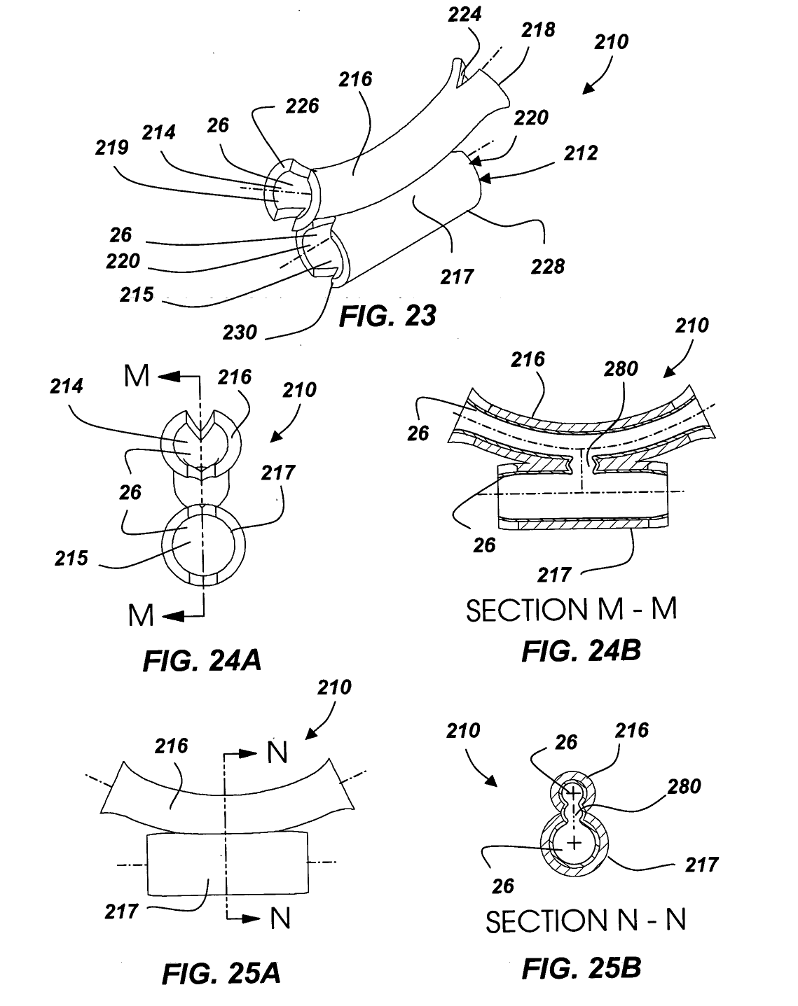

Yet another example embodiment of an anastomotic connector 210 is depicted in FIG. 23. The side-to-side anastomotic connector 210 includes a bio-compatible body 212 having a first tubular portion 216 and a second tubular portion 217. The tubular portions 216, 217 have inner surface contours 214, 215, respectively, with the bio-compatible adhesive 26 applied thereon for connecting to a graft conduit and a primary conduit. The tubular portion 216 has open ends 224 and 226, and the tubular portion 217 has open ends 228 and 230. The inner surface contours 214, 215 include inner circumferential surfaces 218, 219 and 220, 221 adjacent the ends 224, 226 and 228, 230 of the generally tubular portions 216, 217. The inner circumferential surfaces 218, 219 are configured to receive and attach ends of the graft conduit, and inner circumferential surfaces 220, 221 are configured to receive and attach ends of the primary conduit. This anastomotic connector 210 allows for substantially parallel connection of the primary conduit and the graft conduit, or bypass segment, as shown FIG. 24A. The relative location of an anastomotic fenestra 280 is shown in FIGS. 24 and 25, A and B. The open end segments 224, 226, 228, and 230 of the anastomotic connector 210 can be flared, which improves the ease of placement of the biological conduits within the anastomotic connector 210 prior to fixation of the primary conduit and graft or bypass segment with the adhesive 26.

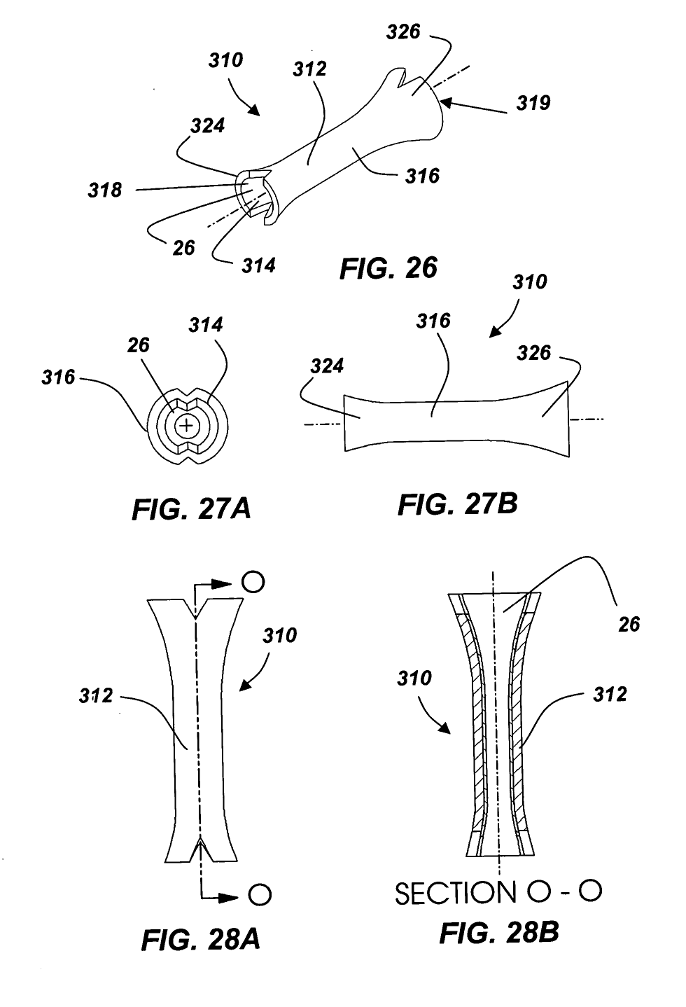

Still another example embodiment of an anastomotic connector 310 is depicted in FIG. 26. The end-to-end anastomotic connector 310 includes a bio-compatible body 312 having a tubular portion 316. The tubular portion 316 has an inner surface contour 314 in which the bio-compatible adhesive 26 can be applied thereon for connecting to a graft conduit and a primary conduit. The tubular portion 316 also has a first end 324 and a second end 326 thereof. The inner surface contour 314 includes inner circumferential surfaces 318, 319 adjacent the ends 324, 326 of the generally tubular portion 316. The inner circumferential surfaces 318, 319 are configured to receive and attach the primary conduit to the graft conduit. In one embodiment, the diameters of the first end 324 and the second end 326 can be substantially equivalent so as to connect biological conduits having diameters of the same size, or ends of the same biological conduit from which an intermediate section has been removed. In another embodiment, the diameter of one end, for example the first end 324, can be smaller in diameter than that of the second end 326, so as to accommodate biological conduits of varying sizes. In this embodiment, the bio-compatible body 312 is preferably gradually tapered in the direction from the second end 326 to the first end 324, as depicted in FIG. 26. The ends 324, 326 are preferably flared to improve placement of the primary and graft conduits prior to fixation with the adhesive 26. In some embodiments, the ends of the primary and graft conduits may overlap, although a flush contact between the primary and graft conduits having square ends is typically more preferable.

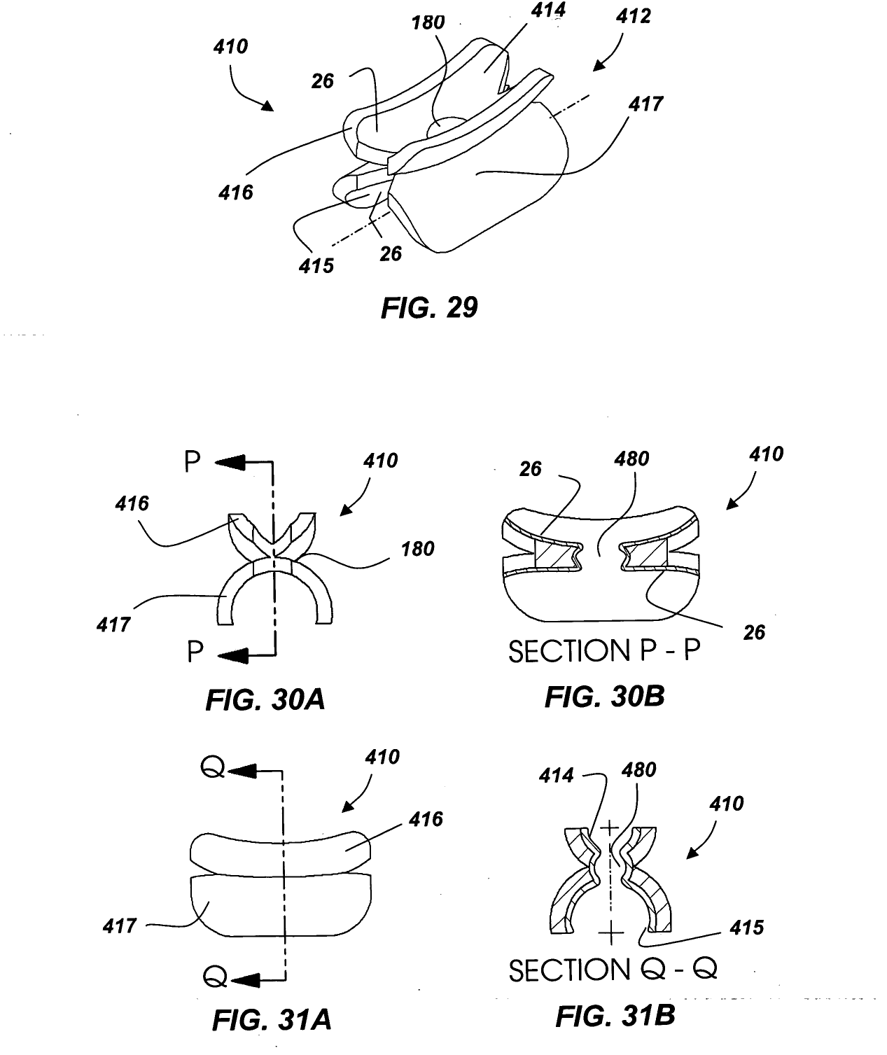

Still another example embodiment of the side-to-side anastomotic connector 410 is presented in FIG. 29. The side-to-side anastomotic connector 410 includes a bio-compatible body 412 having a first flange 416 and a second flange 417. The first flange 416 has an inner surface contour 414 curved in one direction, and the second flange 417 has an inner surface contour 415 curved in the direction away from the curvature of the first flange 416. Bio-compatible adhesive 26 is preferably applied along the inner surface contours 414, 415. This side-to-side anastomotic connector 410 allows for substantially parallel connection of the primary conduit and the graft, or bypass, conduit as shown FIG. 30A. An anatomotic fenestra 480 extends through the flanges 416, 417 to provide fluid communication between the primary and secondary conduits. Flaring of the flanges 416, 417 is preferably provided to improve the ease of placement around the anastomic connector 410 prior to fixation of the primary conduit and graft or bypass segment with the adhesive 26, and to provide a close fit for a more secure seal.

While the invention has been described with reference to preferred and example embodiments, it will be undestood by those skilled in the art that a variety of modifications, additions and deletions are within the scope of the invention, as defined by the following claims.

1. An anastomotic connector for biological conduits, said connector comprising a bio-compatible body having an inner surface contour for attaching an outer surface of a biological conduit therein.

2. The anastomotic connector of claim 1, further comprising a bio-compatible adhesive applied along at least a portion of said inner surface contour.

3. The anastomotic connector of claim 2, wherein the bio-compatible adhesive is a photopolymerizing adhesive.

4. The anastomotic connector of claim 2, wherein the bio-compatible adhesive is activated by contact with a biological fluid.

5. The anastomotic connector of claim 2, wherein the bio-compatible adhesive is activated by exposure to ultra-violet or infra-red light.

6. The anastomotic connector of claim 2, wherein the bio-compatible adhesive comprises microcapsules of adhesive and/or adhesive activators, and the adhesive is activated by rupture of said microcapsules.

7. The anastomotic connector of claim 2, wherein the bio-compatible adhesive comprises a gelatin-formaldehyde-resorcinol glue, a photoethyleneglycol diacrylate, thrombin microcapsules, fibrin microcapsules, a methyl cyanoacrylate, a butyl cyanoacrylates, or combinations thereof.

8. The anastomotic connector of claim 1, wherein at least a portion of said bio-compatible body is translucent.

9. The anastomotic connector of claim 1, wherein said bio-compatible body comprises a polymeric material selected from polytetrafluoroethylene, expanded polytetrafluoroethylene, urethanes, elastomers, polyetherteraphalate, Dacron and combinations thereof.

10. The anastomotic connector of claim 1, wherein said bio-compatible body comprises at least one generally tubular portion, and wherein said inner surface contour comprises an inner circumferential surface adjacent an end of the generally tubular portion of said bio-compatible body.

11. The anastomotic connector of claim 10, wherein the end of the generally tubular portion of said bio-compatible body is flared.

12. The anastomotic connector of claim 10, comprising an end-to-side connector having a generally tubular portion with a first end comprising an inner circumferential surface for receiving and attaching an outer surface of a biological conduit therein, and a second end comprising an attachment flange extending therefrom.

13. The anastomotic connector of claim 10, comprising an end-to-side connector having first and second conduits with an anastomotic fenestra therebetween, the first conduit having a first end with an inner circumferential surface for receiving and attaching a first biological conduit and a second end in communication with the anastomotic fenestra, and the second conduit having first and second ends each with an inner circumferential surface for receiving and attaching a second biological conduit.

14. The anastomotic connector of claim 13, wherein the first and second conduits comprise a generally Y-shaped configuration.

15. The anastomotic connector of claim 10, comprising a side-to-side connector having first and second conduits with an anastomotic fenestra providing fluid communication therebetween, each of the first and second conduits having first and second ends with an inner circumferential surface for receiving and attaching a biological conduit.

16. The anastomotic connector of claim 10, comprising an end-to-end connector comprising a generally tubular conduit having first and second ends, each of the first and second ends comprising an inner circumferential surface for receiving and attaching a biological conduit.

17. The anastomotic connector of claim 1, comprising a side-to-side connector having a first flange with an inner surface contour curved in a first direction, a second flange with an inner surface contour curved in a second direction away from the first direction, and having an anastomotic fenestra extending through the first and second flanges.

18. A method of extravascular anastomosis of at least one biological conduit, said method comprising attaching an exterior surface of the biological conduit to an inner surface of an anastomotic connector.

19. The method of claim 18, wherein the exterior surface of the biological conduit is attached to an inner circumferential surface of a generally tubular portion of the anastomotic connector.

20. The method of claim 18, wherein the biological conduit is attached to the anastomotic connector by a photo-initiated adhesive, and said method further comprises exposing said adhesive to light.

21. The method of claim 18, wherein the biological conduit is attached to the anastomotic connector using a tool having an expandable occlusion feature, and wherein said method further comprises expanding the occlusion feature.

22. The method of claim 18, comprising attaching a first biological conduit to a second biological conduit with an end-to-side anastomotic connector, wherein the exterior surface of an end of the first biological conduit is attached to an inner surface of a generally tubular portion of the connector, and wherein a flange portion of the connector is attached to the second biological conduit.

23. The method of claim 18, comprising attaching a first biological conduit to a second biological conduit with an end-to-side anastomotic connector, wherein the exterior surface of an end of the first biological conduit is attached to an inner surface of a first generally tubular portion of the connector, and wherein exterior surfaces of ends of the second biological conduit are attached to inner surfaces of a second generally tubular portion of the connector.

24. The method of claim 18, comprising attaching a first biological conduit to a second biological conduit with a side-to-side anastomotic connector, wherein exterior surfaces of first and second ends of the first biological conduit are attached to inner circumferential surfaces at first and second ends of a first generally tubular portion of the connector, and wherein exterior surfaces of first and second ends of the second biological conduit are attached to inner circumferential surfaces at first and second ends of a second generally tubular portion of the connector.

25. The method of claim 18, comprising attaching a first biological conduit to a second biological conduit with a side-to-side anastomotic connector, wherein an exterior surface of the first biological conduit is attached to a first flange of the connector, and wherein an exterior surface of the second biological conduit is attached to a second flange of the connector, said connector comprising an anastomotic fenestra through the first and second flanges.

26. The method of claim 18, comprising attaching a first end of a biological conduit to a second end of a biological conduit with an end-to-end anastomotic connector, wherein a first end of a biological conduit is attached to an inner surface at a first end of the connector, and a second end of a biological conduit is attached to an inner surface at a second end of the connector.

27. The method of claim 18, further comprising applying an adhesive to the inner surface of the anastomotic connector.

28. A tool for anastomosis and connection of biological conduit(s), said tool comprising:
a shaft having a sharp tip for penetrating tissue of a biological conduit; and
an expanding portion for engaging the biological conduit.

29. The tool of claim 28, wherein the expanding portion comprises an inflatable member, and wherein the shaft comprises at least one lumen for communicating fluid for inflation of said inflatable member.

30. The tool of claim 28, wherein the shaft is flexible.

31. The tool of claim 30, wherein the shaft is steerable.

32. The tool of claim 28, further comprising a light source.

33. The tool of claim 28, further comprising endoscopic imaging means.

34. The tool of claim 28, wherein the shaft comprises an inner shaft having a cutting head comprising the sharp tip mounted thereon, the tool further comprising an outer shaft to which the expanding portion is mounted, and having a cutting collar attached to the outer shaft between the expanding portion and the cutting head, whereby the inner shaft is slidable relative to the outer shaft to advance and retract the cutting head.
